(19) United States
(12) Patent Application Publication
(10) Pub. No.: US 2005/0181034 A1
(43) Pub. Date: Aug. 18, 2005

(54) Formulation of liposomal derivatives of phenylalanine

(75) Inventors: Katja Wosikowski-Buters, Poing (DE); Wolfgang Schmalix, Grobenzell (DE)

Correspondence Address: ROTHWELL, FIGG, ERNST & MANBECK, P.C., 1425 K STREET, N.W., SUITE 800, WASHINGTON, DC, 20005, US

(73) Assignee: WILEX AG, Munchen (DE)

(21) Appl. No.: 10/521,805

(22) PCT Filed: Jul. 22, 2003
(86) PCT No.: PCT/EP03/08011

(30) Foreign Application Priority Data
Jul. 24, 2002 (DE) 102 33 632.6

(51) Int. Cl. A61K 31/495; A61K 9/127

(52) U.S. Cl. 424/450; 514/255.01

(57) ABSTRACT

The invention relates to pharmaceutical formulations of phenylalanine derivatives and to the use thereof as urokinase inhibitors, in particular for the treatment of malignant tumors and of tumor metastases.

The invention relates to pharmaceutical formulations of phenylalanine derivatives and to the use thereof as urokinase inhibitors, in particular for the treatment of malignant tumors and of tumor metastases.

The ability of solid tumors to spread and metastasize into surrounding tissue correlates with the breakdown or transformation of the extracellular matrix (tumor stroma) in the surroundings of the tumor cell and with their ability to penetrate the basement membrane. Although the (patho)biochemical relationships have not yet been definitively explained, a central significance is ascribed to the plasminogen activator urokinase (uPA) and the urokinase receptor (uPAR). uPA mediates the proteolytic cleavage of plasminogen to plasmin. Plasmin in turn is a protease with a wide range of effects which is able to break down components of the extracellular matrix such as fibrin, fibronectin, laminin and the protein framework of the proteoglycans directly. In addition, plasmin can activate “latent” metalloproteases and the inactive proenzyme of uPA, pro-uPA.

Tumor cells and nonmalignant cells of the tumor stroma synthesize and secrete the enzymatically inactive proenzyme pro-uPA. Proteases such as, for example, plasmin or cathepsins B and L cleave pro-uPA by limited proteolysis to the active serine protease HMW-uPA (HMW=high molecular weight). pro-UPA and the active protease HMW-uPA bind to the cell surface receptor uPAR (CD87). Plasmin(ogen) likewise binds to specific receptors on the plasma membrane of the tumor cell, thus achieving focusing and amplification of plasminogen activation in the immediate surroundings of the tumor cell. Invasive cells are thus enabled to break down the extracellular matrix without evading the bases necessary for directed movement through proteolysis.

It has been possible to show in various cytological studies that the cell-associated plasminogen activator system occupies a special position within the cascade-like reaction pathways of tumor-associated proteolysis systems (Wilhelm et al. (1994) The Urokinase/Urokinase receptor system: A new target for cancer therapy? In: Schmitt M., Graeff H., Kindermann G. (Ed.): Prospects in Diagnosis and Treatment of Cancer. International Congress Series, Excerpta Medica 1050, Amsterdam, Elsevier 1994, pp 145-156). It was observed on cultures of human colon carcinoma cells that their ability to migrate through an extracellular matrix depends on the degree of saturation of the uPA receptors with active uPA (Hollas et al., Cancer Res. 51 (1991), 3690-3695). Likewise in a cell culture model there was observed to be a reduction in the invasive potential of cells when the proteolytic activity of uPA was inhibited by PAI-1 (Cajot et al., Proc. Natl. Acad. Sci. USA 87 (1990), 6939-6943) or PAI-2 (Baker et al., Cancer Res. 50 (1990), 4676-4684). A comparable effect was achieved on inhibition of the binding of uPA to the cell surface by blocking the receptor by means of proteolytically inactive uPA variants (Cohen et al., Blood 78 (1991), 479-487; Kobayashi et al., Br. J. Cancer 67 (1993), 537-544). Transfection of epidermoid carcinoma cells with a plasmid which expresses an antisense transcript against a part of uPAR also led, through suppression of uPAR synthesis, to a reduction in the invasiveness of these cells (Kook, EMBO J. 13 (1994), 3983-3991). Antibodies directed against uPA and PAI-1 reduced the invasive potential of lung cancer cells in vitro (Liu et al., Int. J. Cancer 60 (1995), 501-506).

It has also been possible to demonstrate the influence of the plasminogen activator system on the metastatic process in tumor animal models. Thus, the formation, caused by human carcinoma cells, of pulmonary metastases in chicken embryos were almost completely prevented by adding antibodies against uPA (Ossowski and Reich, Cell 35 (1983), 611-619). Metastatic human carcinoma cells were transfected with an expression plasmid which coded for a proteolytically inactive but uPAR-binding uPA mutant. It was shown in a mouse model that the carcinoma cells which synthesized inactive uPA formed a significantly smaller number of metastases after injection compared with the untransfected cells (Crowley et al., Proc. Natl. Acad. Sci. USA 90 (1993), 5021-5025). In addition, administration of uPA antisense oligonucleotides was observed to be followed by an inhibition of the intraperitoneal dissemination of human ovarian carcinoma cells in nude mice (Wilhelm et al., Clin. Exp. Metast. 13 (1995), 296-302).

In recent years, the clinical relevance of factors of the plasminogen activator system (uPA, uPAR, PAI-1 and PAI-2) for the prognosis of patients with solid malignant tumors has been intensively investigated. This has revealed that the uPA antigen content in various tumors (e.g. breast, ovary, stomach, lung, kidney etc.) is a strong prognostic factor both for recurrence-free survival and for mortality (see, for example, Schmitt et al., J. Obstet. Gynaecol. 21 (1995), 151-165; Jaenicke et al., Breast Cancer Res. Treat. 24 (1993), 195-208; Kuhn et al., Gynecol. Oncol. 55 (1994), 401-409; Nekarda et al., Lancet 343 (1994), 117; Pedersen et al., Cancer Res. 54 (1994), 4671-4675). Likewise, elevated concentrations of uPAR in lung cancer tissue (Pedersen et al., supra) and breast cancer tissue (Duggan et al., Int. J. Cancer 61 (1995), 597-600; Ronne et al., Breast Cancer Res. Treat. 33 (1995), 199-207) and in stomach cancer, both in the tumor tissue itself (Heiss et al., J. Clin. Oncol. 13 (1995), 2084-2093) and in the tumor cells scattered in the bone marrow (Heiss et al., Nature Medicine 1 (1995), 1035-1039) correlate with a poor prognosis.

It has also been found that 3-amidinophenylalanine derivatives substituted in position 2 by a phenyl radical are selective uPA inhibitors which are active in vivo (PCT/EP99/05145). These compounds are administered in animal experiments in the form of aqueous solutions. DE 102 25 876.7 discloses the use of 3-guanidinophenylalanine derivatives as urokinase inhibitors.

It has emerged from the first clinical trials of the abovementioned compounds that administration in the form of aqueous solutions is associated with disadvantages. Thus, it has been found that the compounds have hemolytic properties on intravenous injection or infusion of relatively highly concentrate solutions, and lead to skin irritation on subcutaneous administration. However, large infusion volumes are necessary to administer solutions of low concentration, and subcutaneous administration of effective amounts of the agent in the form of aqueous solutions is not possible.

There was thus a need to develop novel pharmaceutical formulations of the phenylalanine derivatives which, on the one hand, are stable and have high activity but, on the other hand, do not lead to unwanted side effects such as hemolysis or skin irritation.

However, attempts to stabilize aqueous solutions by adding surface-active agents such as, for example, Pluronic F68 and Tween 80, or stabilizers such as human serum albumin, were unsuccessful. Nor did addition of cosolvents such as polyethylene glycols lead to the desired result. Finally, it was not possible to provide sufficient stability by formulating the active ingredient in mixed micelles comprising the bile salt glycocholate monohydrate and the phospholipid egg phosphatidylcholine, either.

The object on which the invention is based was thus to provide pharmaceutical formulations with a urokinase inhibitor derived from 3-amidinophenylalanine or 3-guanidinophenylalanine, which, on the one hand, have high activity and, on the other hand, are stable and tolerated.

The present invention relates in particular to novel urokinase inhibitors derived from 3-amidinophenylalanine or 3-guanidinophenylalanine, of the general formula I 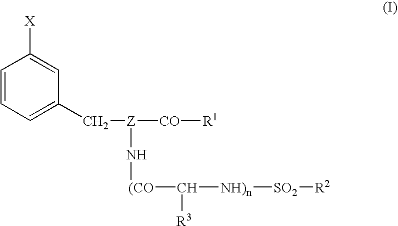
which are in the form of racemates and as compounds having the L or D configuration, and in which
X is an amidino or guanidino group,
R1 (a) is OH or OR⁴, where R⁴ is an optionally substituted, e.g. by hydroxyl, carboxyl, sulfonyl, nitro, cyano, oxo or/and halogen, branched or unbranched C₁-C₈-alkyl, C₃-C₈cycloalkyl or aralkyl, e.g. benzyl or phenylethyl,
   (b) is a group of the formula 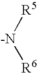
   in which R⁵ and R⁶ are any radicals compatible with the overall structure, where in particular
      (i) R⁵ and R⁶ are H,
      (ii) R⁵ is H, and R⁶ is an optionally substituted, e.g. by hydroxyl, carboxyl, sulfonyl, nitro, cyano, oxo or/and halogen, branched or unbranched C₁-C₈ alkyl, aralkyl, e.g. benzyl or phenylethyl, or C₅-C₈ cycloalkyl,
      (iii) R⁵ and R⁶ are each independently an optionally substituted, e.g. by hydroxyl or/and halogen, unbranched or branched C₁-C₄ alkyl, or
      (iv) R⁵ is H, and R⁵ is —NH₂ or an in particular aryl- or heteroaryl-substituted amino group,
      (v) R⁵ is H or an optionally substituted, e.g. by hydroxyl or/and halogen, unbranched or branched C₁-C₄ alkyl, and R⁶ is the residue of an amino acid, e.g. of an α-, β- or ω-amino carboxylic or amino sulfonic acid, or the residue of a peptide, e.g. having a length of up to 50 amino acids, or of a polypeptide, e.g. having a length of more than 50 amino acids and up to 1000 amino acids,
   (c) is a group of the formula 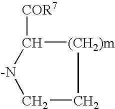
   in which m is the number 1 or 2, and in which one or more of the methylene groups are optionally substituted, e.g. by a hydroxyl, carboxyl, C₁-C₄-alkyl or aralkyl radical, e.g. benzyl or phenylethyl, where the group (c) is racemic or has the D or L configuration, and R⁷ has the meaning of R¹ in sections (a), (b) and (f),
   (d) is a group of the formula 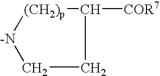
   in which p=r=1, p=1 and r=2 or p=2 and r=1, and in which one or more of the methylene groups are optionally substituted, e.g. by a hydroxyl, carboxyl, C₁-C₄-alkyl or aralkyl radical, e.g. benzyl or phenylethyl, and R⁷ has the meaning of R¹ in section (a), (b) and (f),
   (e) is a piperidyl group which is optionally substituted in one of positions 2, 3 and 4, e.g. by a C₁-C₄-alkyl, C₁-C₃-alkoxy or hydroxyl radical, where a further aromatic or cycloaliphatic ring, preferably phenyl or cyclohexyl, is optionally fused onto the heterocycloaliphatic rings of the formulae (c), (d), (e) in the 2, 3 or 3,4 position relative to the heteroatom,
   (f) is a group of the formula 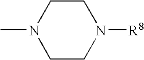
      in which R⁸ is
      (i) an optionally substituted, e.g. by C₁-C₆-alkyl, C₁-C₃-alkoxy, hydroxyl, carboxyl, sulfonyl, nitro, cyano, oxo or/and halogen, C₁-C₆-alkyl radical, such as, for example, ethoxycarbonyl, or aryl radical, such as, for example, phenyl, p-halophenyl, naphthyl,
      (ii) a saturated or unsaturated, branched or unbranched C₁-C₆-alkoxy radical or
      (iii) an optionally substituted, e.g. by C₁-C₆-alkyl, C₁-C₃-alkoxy, hydroxyl, carboxyl, sulfonyl, nitro, cyano, oxo or/and halogen, phenoxy- or benzyloxycarbonyl radical,
   (g) is an acyl radical of the formula —COX, where X is
      (i) H, an optionally substituted, e.g. by hydroxyl, carboxyl, sulfonyl, nitro, cyano, oxo or/and halogen, unbranched or branched alkyl radical, preferably a C₁-C₆-alkyl radical, in particular methyl,
      (ii) an optionally substituted, e.g. by C₁-C₆-alkyl, C₁-C₃-alkoxy, hydroxyl, carboxyl, sulfonyl, nitro, cyano, oxo or/and halogen, aryl or heteroaryl radical such as, for example, phenyl, p-halophenyl, thienyl or
      (iii) an optionally substituted e.g. by hydroxyl, carboxyl, sulfonyl, nitro, cyano, oxo or/and halogen, cycloalkyl radical, preferably a C₃-C₁₀-cycloalkyl radical,
   (h) is an aralkyl radical, e.g. benzyl or phenylethyl, in which the aromatic radical is optionally substituted, e.g. by a halogen atom, a C₁-C₆-alkyl, C₁-C₃-alkoxy, hydroxy, cyano, carboxyl, sulfonyl or nitro group,
   (i) is a carboxamide residue of the formula —CONR′R″, a thiocarboxamide residue —CSNR′R″ or an acetamide residue —CH₂—CONR′R″, where
      (i) R′ and R″ are H,
      (ii) R′ and R″ are each independently C₁-C₄-alkyl,
      (iii) R′ is H and R″ is C₁-C₄-alkyl,
      (iv) R′ is H and R″ is aryl, e.g. phenyl, or
      (v) R′ and R″ form with the nitrogen atom a heterocycloaliphatic ring having 5-7 ring members, which may include a further heteroatom, e.g. N, O or/and S,
   (j) is an SO₂—Y radical in which Y is
      (i) an optionally substituted, e.g. by hydroxyl, carboxyl, sulfonyl, nitro, cyano, oxo or/and halogen, C₁-C₈-alkyl, preferably methyl, trifluoromethyl, trichloromethyl,
      (ii) an optionally substituted, e.g. by C₁-C₆-alkyl, C₁-C₃-alkoxy, hydroxyl, carboxyl, sulfonyl, nitro, cyano, oxo or/and halogen, aryl or heteroaryl such as, for example, phenyl, 4-methylphenyl, 2,4,6-trimethylphenyl, 2,4,6-triisopropylphenyl, 4-methoxy-2,3,6-trimethylphenyl, 2,2-dimethyl-6-methoxychromanyl, 2,2,5,7,8-pentamethylchromanyl, anthraquinonyl, naphthyl or quinolyl, or O-aryl, preferably O-phenyl, or O-heteroaryl or
      (iii) —NR′R″, where R′ and R″ are each independently H or C₁-C₃-alkyl,
   (k) is a cycloaliphatic ring having 5 to 8 C atoms, which is optionally substituted, e.g. by a C₁-C₆-alkyl, C₁-C₃-alkoxy, halogen, hydroxyl or/and oxo group,
   (1) is an optionally substituted, e.g. by C₁-C₆-alkyl, C₁-C₃-alkoxy, hydroxyl, carboxyl, sulfonyl, nitro, cyano, oxo or/and halogen, heteroaryl radical such as, for example, pyridyl or pyrimidyl, or heterocycloaliphatic radical, for example N-methylpiperidyl,
   (m) is a functionalized alkyl radical of the formula —(CH₂)ₙ—X, where the alkyl chain is unbranched or branched, n is 1 to 8, and the functional radical X
      (i) is a hydroxyl group whose H atom is optionally replaced by a C₁-C₄-alkyl, aralkyl, e.g. benzyl or phenylethyl, aryl, e.g. phenyl, C₁-C₄-hydroxyalkyl or acyl group CO-alkyl, (C₁-C₆),
      (ii) is a halogen atom,
      (iii) is a tertiary amino group of the formula —N(Alk)₂, where the alkyl groups have 1 to 3 C atoms and preferably the same meaning, and the nitrogen atom optionally belongs to a heterocycloaliphatic ring having 5-7 ring members, which may include a further heteroatom, e.g. N, O or/and S.
R² is an optionally substituted, e.g. by C₁-C₆-alkyl, C₁-C₃-alkoxy, hydroxyl, carboxyl, sulfonyl, nitro, cyano, oxo or/and halogen, phenyl radical such as, for example, phenyl, 4-methylphenyl, 2,4,6-trimethylphenyl, 2,4,6-triisopropylphenyl, 4-methoxy-2,3,6-trimethylphenyl,
R³ is H or branched or unbranched C₁-C₄-alkyl, and n is 0 or 1,
Z is N or CR⁹, where R⁹ is H or branched or unbranched C₁-C₄-alkyl.

The compounds may also be in the form of salts, preferably physiologically tolerated acid salts, e.g. salts of mineral acids, particularly preferably hydrochlorides, or salts of suitable organic acids.

Of the compounds defined in the general claims, those in which R¹ corresponds to a group of the formulae (b), (d) and (f), R² is a mono-, bi- or tri-alkyl-substituted phenyl radical, in particular a 2,4,6-substituted phenyl radical, e.g. a 2,4,6-triisopropylphenyl radical, and n is 0, are particularly important. Compounds in which Z is CH or N are further preferred.

The compound of the formula (I) is particularly preferably Nα-(2,4,6-triisopropylphenylsulfonyl)-3-amidino-(D,L)-phenylalanine 4-ethoxycarbonylpiperazide, Nα-(2,4,6-triisopropylphenylsulfonyl)-3-guanidino-(D,L)-phenylalanine 4-ethoxycarbonylpiperazide or the L enantiomer thereof or a pharmaceutically acceptable salt of these compounds.

The compounds of the invention are able to inhibit highly efficiently the growth or/and the dissemination of malignant tumors, e.g. tumor dissemination in cases of pancreatic carcinoma, tumor growth of carcinoma of the breast, and the metastasis of tumors. The uPA inhibitors can moreover be employed where appropriate together with other antitumor agents or with other modes of treatment, e.g. radiation or surgical procedures. In addition, the inhibitors of the invention are also effective for other uPA-associated disorders (e.g. for preventing the formation of blisters associated with the skin disease pemphigus vulgaris).

The liposomal formulations of the invention comprise the active ingredient preferably in a proportion by weight of 0.5-10%, particularly preferably of 2-5%, based on the total weight of the formulation. It is further preferred for the formulation—at least its aqueous component—to have a pH in the range 5.5-9.0. The content of free active ingredient, i.e. that present after removal of lipid components by filtration in the aqueous phase of the formulation, is preferably ≦1 mg/ml, particularly preferably ≦500 μg/ml and most preferably ≦100 μg/ml.

The formulation preferably comprises phospholipids in a proportion by weight of 4.5-40%, particularly preferably of 6-15%, based on the total weight of the formulation. Examples of suitable phospholipids are neutral phospholipids such as, for example, phosphatidylcholine, anionic phospholipids such as, for example, phosphatidylethanolamine, phosphatidylglycerol, diphosphatidylglycerol (cardiolipin), phosphoinositol and esterified derivatives thereof, such as, for example, dimyristoylphosphatidylglycerol. It is possible to use for example phospholipids from natural sources, e.g. egg or soybean lecithin, synthetic phospholipids or combinations thereof. The formulation particularly preferably comprises at least one anionic phospholipid. Good results have been obtained on use of a combination of phosphatidylcholine and dimyristoylphosphatidylglycerol in a ratio of 70:30 by weight.

In addition, the formulation may preferably comprise a membrane-stabilizing component such as, for example, cholesterol or derivatives thereof, in a proportion by weight of up to 5% based on the total weight of the formulation. The membrane stability or rigidity can, however, also be adjusted by the selection of appropriate fatty acids in the phospholipids or, where appropriate, other lipid components via the chain length or/and the degree of unsaturation.

The formulation preferably also comprises a cryoprotectant which is beneficially present in a proportion by weight of up to 15%, preferably of 5-15%, based on the total weight of the formulation. Examples of suitable cryoprotectants are carbohydrates, e.g. mono-, di- or trisaccharides, such as, for example, lactose, sucrose, trilactose, maltose, trehalose etc. or/and sugar alcohols such as, for example, sorbitol, mannitol etc.

The liposomal formulation of the invention may comprise unilamellar liposomes, multilamellar liposomes, unordered complexes and combinations thereof. Preferred liposomes have an average diameter after production not greater than 500 nm and, in particular, in the range 100-250 nm.

The liposomal formulation of the invention can be produced for example by high-pressure homogenization of a suspension comprising the active ingredient and the lipids and, where appropriate, subsequent filtration. Other possibilities are known to the skilled worker, e.g. solvent injection, hydration of lipid films etc.

The liposomal formulation can be sterilized by filtration and, where appropriate, dehydrated, e.g. by lyophilization. The average diameter may increase, e.g. to 100-1000 nm, after aqueous reconstitution of dehydrated liposomes.

The formulation of the invention can be employed for parenteral administration, e.g. for intravenous injection, for infusion or for subcutaneous or intramuscular injection. The daily dose is preferably 5-250 mg, particularly preferably 20-120 mg on subcutaneous or intramuscular administration and 10-500 mg, particularly preferably 50-250 mg on intravenous administration, in each case based on an average body weight of 70 kg. Administration preferably takes place once a day to once a week.

The formulation of the invention can be employed where appropriate in combination with other active ingredients, e.g. cytoxic agents. Joint administration is preferred, e.g. as treatment before or/and after surgical procedures, radiotherapy or/and chemotherapy.

The invention is further to be explained in more detail by the following examples.

### EXAMPLE 1

### Production of Liposomal Preparations

1.1 Materials

Dimyristoylphosphatidylglycerol sodium salt (DMPG-Na) was purchased from Nippon Fine Chemicals. Egg phosphatidylcholine (EPC) was purchased from Lipoid KG.

1.2 Production of Liposomes with pH 6.5

The phospholipids and the active ingredient Nα-(2,4,6-triisopropylphenylsulfonyl)-3-amidino-(L)-phenylalanine 4-ethoxycarbonylpiperazide (WX-UK1) as hydrochloride were dispersed together in 50 mmol/l phosphate buffer of pH 6.5 and 9% lactose. The mixture was then heated to 40° C. and put in an ultrasonic bath for 30 min.

An EmulsiFlex C5 apparatus from Avestin, equipped with an extrusion unit, was used for the high-pressure homogenization so that it was possible to carry out homogenization and high-pressure extrusion through a polycarbonate membrane filter in series.

Firstly, the liposome suspension was passed through the high-pressure homogenizer for 6 min (corresponding to 20 passages) under a pressure of 345 bar. The formulation was then passed for 6 min through a 100 nm polycarbonate membrane filter in the extrusion unit.

1.3 Production of Liposomes with pH 8.4

Liposomal preparations with a pH of 8.4 were obtained in a similar way. Firstly, however, a dispersion with a pH of 5.2 was prepared and subjected to a high-pressure homogenization. The pH was then adjusted to 8.4 by adding disodium hydrogen phosphate, and a further homogenization was carried out for 6 min under a pressure of 345 bar.

1.4 Sterilization by Filtration, Product Filling and Lyophilization

The liposomal formulations were filtered through a sterilized 450 nm filter (Durapore polyvinylidene difluoride from Millipore), and the filtered suspensions were filled in 0.5 ml aliquots and then lyophilized.

1.5 Size Determination

The average size and the size distribution of the liposomal suspensions were determined using a Malvern Zetasizer 1000. The degree of homogeneity of the suspensions has been expressed as polydispersity index (PI). A value of 0.1 means a very narrow size distribution and a value of 0.9 a very wide distribution.

1.6 Results

The compositions and the results of analysis are summarized in Table 1 below. After reconstitution, the active ingredient content of the liposomal formulation was between 18 and 21 mg/ml and the particle size was between 350 and 500 nm, there having been a significant increase in the particle size during the lyophilization and reconstitution.

Liposomes which comprise the active ingredient together with negatively charged phospholipids were dilutable, whereas neutral liposomes showed precipitation on addition of buffer.

TABLE 1

| | | | | Actual |
|---|---|---|---|---|
| | | | | concen- |
| | | | | tration |
| | | | | after |
| | Concen- | Particle size (nm)/PI | | recon- |
| | tration | before | after | stitution |
| Components | (%) | reconstitution | reconstitution | (%) |
| Formulation 1 | | | | |
| EPC pH 6.5 | | | | |
| WX-UK1.HCl | 2.00 | 108/0.27 | 518/0.53 | 1.98 |
| EPC | 10.00 | | | |
| Lactose | 7.91 | | | |
| Na₂HPO₄ × 2H₂O | 0.72 | | | |
| Water | ad 100% | | | |
| Formulation 2 | | | | |
| PC/PG pH 6.5 | | | | |
| WX-UK1.HCl | 2.00 | 307/0.49 | 405/0.45 | 2.05 |
| EPC | 7.00 | | | |
| DMPG.Na | 3.00 | | | |
| Lactose | 7.90 | | | |
| Na₂HPO₄ × 2H₂O | 0.72 | | | |
| Water | ad 100% | | | |
| Formulation 3 | | | | |
| EPC pH 8.4 | | | | |
| WX-UK1.HCl | 2.00 | 269/0.24 | 359/0.34 | 1.80 |
| EPC | 10.00 | | | |
| Lactose | 9.70 | | | |
| Na₂HPO₄ × 2H₂O | 0.70 | | | |
| Water | ad 100% | | | |
| Formulation 4 | | | | |
| PC/PC pH 8.4 | | | | |
| WX-UK1.HCl | 2.00 | 202/0.23 | 353/0.43 | 1.98 |
| EPC | 7.00 | | | |
| DMPG.Na | 3.00 | | | |
| Lactose | 9.70 | | | |
| Na₂HPO₄ × 2H₂O | 0.40 | | | |
| Water | ad 100% | | | |

### EXAMPLE 2

### Bioavailability and Tolerability of Liposomal Formulations in Rats

2.1 Administration

Female wistar rats, 240-300 g body weight (Charles River-Wiga, Sulzfeld), were treated with the active ingredient WX-UK1 in the form of an aqueous solution or with the liposomal formulations 1-4 described in Example 1. The animals each received 1 dose/day over a period of 10 days. The administrations took place in the region of the flank (between shoulder and rear limbs) so that the animals were unable to reach the injection site with their paws. The injection sites were approx. 1 cm apart, no site being injected more than once. The first five injections took place on the left side, and the second five injections on the right side. The dose was usually 3 mg of active ingredient/kg of body weight, i.e. 0.9 mg of active ingredient was administered for an average body weight of 300 g. Radiolabeled active ingredient (3H-WX-UK1) was administered in some cases.

2.2 Observation of the Animals

The animals were weighed before each injection and observed for 2 h after each injection, and before each further injection the previous injection sites were palpated. The behavior of the animals and the palpation findings were recorded.

The following score was used to evaluate the observed impairments:

Behavior of the animals after the injection (2 h)

| | |
|---|---|
| normal | 0 |
| slight scratching in 1^{st} hour | 1 |
| scratching and signs of pain | 2 |
| still marked impairment after 2 h | 3 |

Consecutive Palpation Findings

| | |
|---|---|
| unremarkable | 0 |
| slight, isolated thickenings/indurations | 1 |
| persistent thickenings/indurations, coalescing | 2 |
| additional lesions, inflammations | 3 |

2.3 Investigations after Termination of Administration

24 h after the last injection, the animals were anaesthetized (ethylurethane, 1.4 g/kg i.p.), and citrated blood was obtained from the retroorbital venous plexus; citrated plasma was obtained by centrifugation at 1200×g for 10 min. The bile duct was then dissected, and bile was obtained for 15 min. Subsequently, the areas of skin at the injection sites were examined and the growth findings on dissection were recorded.

The following score was used to evaluate the growth changes observed:

Final Observations, Growth Findings during Dissection

| | |
|---|---|
| unremarkable | 0 |
| isolated indurations or foci of thickening | 1 |
| numerous foci of thickening or nodes | 2 |
| numerous foci of thickening and inflammation | 3 |
| or nodes | |

Skin samples were dissected from the regions of the injections, fixed in formalin and examined histologically (hematoxylin/eosin stain). The following score was used to evaluate the histologically visible changes:

Histological Findings:

| | |
|---|---|
| unremarkable | 0 |
| isolated necroses, isolated inflammatory | 1 |
| reactions | |
| necroses or inflammatory reactions | 2 |
| necroses and strong inflammatory reactions | 3 |
| and/or further changes | |

The animals were then sacrificed, and various organs (heart, kidney, liver, spleen) were dissected. The active ingredient content in the plasma, bile and organs was determined by HPLC [standard method using Nucleosil 7 C18 columns (Macherey-Nagel, Düren), acetonitrile/water/perchloric acid 30/70/0.04, 1 ml/min] after prepurification using Chromabond C18 solid-phase extraction columns (Macherey-Nagel, Duren). When ³H-WX-UK1 was used, the active ingredient concentration was determined immediately after lyophilization of the samples by measuring the radioactivity in a liquid scintillation counter.

2.4 Results

Administration of WX-UK1 dissolved in 0.5 ml of NaCl (0.9%) with 5% ethanol (experiments 628, 629) served as controls.

Lyophilized neutral liposomes of pH 6.5 (formulation 1) were resuspended with water and employed directly. In accordance with the concentration of 20 mg/ml which was then present, 45 μl were administered per rat (300 g body weight), which corresponds to the desired dose of 3 mg/kg of body weight (experiments 624, 625).

Lyophilized negative liposomes of pH 6.5 (formulation 2) were resuspended with water. In accordance with the concentration of 16 mg/ml then present, 55 μl were administered per rat (300 g body weight), which corresponds to the desired dose of 3 mg/kg of body weight (experiments 626, 627).

Lyophilized neutral liposomes of pH 8.4 (formulation 3) were resuspended with water and employed directly. In accordance with the concentration of 20 mg/ml which was then present, 45 μl were administered per rat (300 g body weight), which corresponds to the desired dose of 3 mg/kg of body weight (experiments 632, 633).

Lyophilized negative liposomes of pH 8.4 (formulation 4) were resuspended with water and employed directly. In accordance with the concentration of 20 mg/ml then present, 45 μl were administered per rat (300 g body weight), which corresponds to the desired dose of 3 mg/kg of body weight (experiments 637, 638).

2.5 Summary of the Results

Table 2 contains the concentrations of WX-UK1 in plasma, bile and the organs kidney, liver, spleen and heart. The concentration in the plasma could be determined only by using ³H-WX-UK1 and is <100 ng/ml. Very comparable concentrations are found in the controls with administrations in NaCl/ethanol and administration of liposomes 3 and 4. Lower concentrations of WX-UK1 were found in bile and organs with liposomes 1 and 2. Table 2 is a compilation of the assessment of the histological findings.

Tables 3 and 4 contain the assessment of the consequences of administration to the animals. It is evident that administration of the liposomal formulations leads to considerably milder side effects than administration of the aqueous solution.

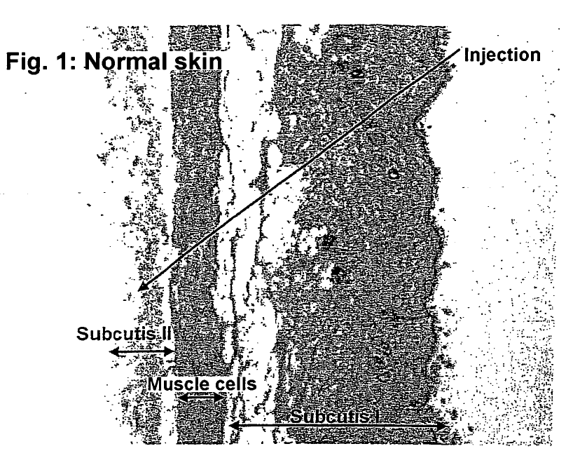

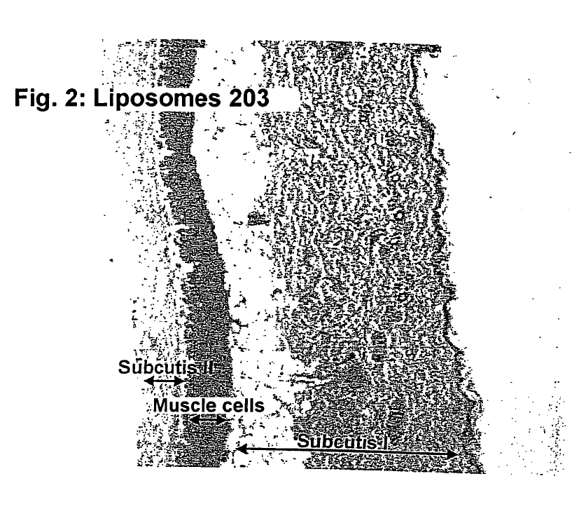

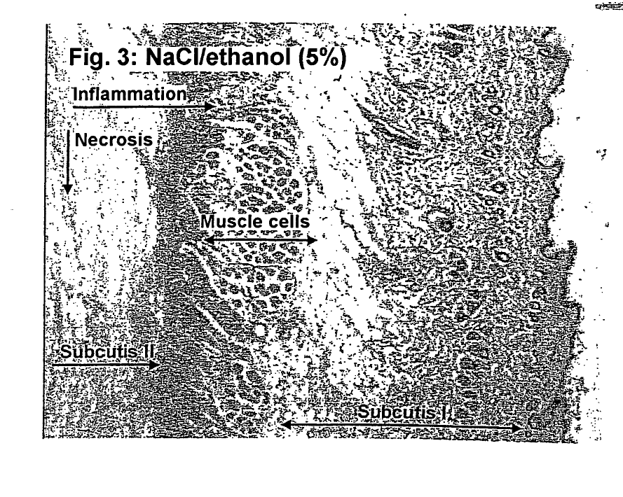

FIG. 1 (magnification 25×) depicts skin after administration after WX-UK1 dissolved in liposomes 4. No changes from normal skin (FIG. 2) are evident.

FIG. 3 (magnification 25×) shows the findings after administration of WX-UK1 in NaCl/ethanol. A strong inflammatory reaction is evident in the margin between subcutis II and the muscle cells, and subcutis II shows necrotic distension.

TABLE 2
Concentration in plasma, bile and organs
after s.c. administration of various
preparations (n.n. = not determined)

| | | Concentration | | | | | |
|---|---|---|---|---|---|---|---|
| Ex. | | (μg/ml) | | Organ content (μg/g) | | | |
| No. | Administration | Plasma | Bile | Kidney | Liver | Spleen | Heart |
| 628 | Control | <0.05 | 3.1 | 2.5 | 0.36 | 0.65 | 1.0 |
| 629 | Control | <0.05 | 3.1 | 2.3 | 0.18 | 0.69 | 0.84 |
| 624 | Liposomes 1 | <0.05 | 2.7 | 1.9 | 0.18 | 0.46 | 0.31 |
| 625 | Liposomes 1 | <0.05 | 4.2 | 1.5 | 0.48 | 0.44 | 0.88 |
| mean | | <0.05 | 3.4 | 1.7 | 0.33 | 0.45 | 0.60 |
| 626 | Liposomes 2 | <0.05 | 1.6 | 0.66 | 0.23 | 0.23 | 0.31 |
| 627 | Liposomes 2 | <0.05 | 2.5 | 1.7 | 0.35 | 0.14 | 0.32 |
| mean | | <0.05 | 2.05 | 1.2 | 0.29 | 0.18 | 0.32 |
| 632 | Liposomes 3 | n.d. | 7.2 | 4.12 | 0.90 | 0.40 | 1.74 |
| 633 | Liposomes 3 | n.d. | 6.85 | 2.49 | 0.31 | 0.49 | 0.66 |
| mean | | | 7.0 | .030 | 0.60 | 0.44 | 1.20 |
| 637 | Liposomes 4 | n.d. | 6.78 | 2.92 | 0.36 | 0.38 | 1.09 |
| 638 | Liposomes 4 | n.d. | 9.8 | 3.49 | 0.78 | 0.48 | 1.19 |
| mean | | | 8.3 | 3.20 | 0.57 | 0.43 | 1.50 |

TABLE 3
Histological assessment of the examined areas
of skin

| No. | Administration | Score | Findings |
|---|---|---|---|
| 628 | Control | 3 | circumscribed necroses with |
| | | | inflammatory marginal |
| | | | reaction (leukocytes, |
| | | | incipient granulation tissue) |
| 629 | Control | 3 | as 629 |
| | Mean = | 3 | |
| 624 | Lip. 1 | 0 | nothing found/no histo |
| 625 | Lip. 1 | 1 | only 1 focus: necroses and |
| | | | pronounced marginal reaction |
| | | | (as contr.) |
| | Mean = | 0.5 | |
| 626 | Lip. 2 | 1 | 2 small foci: inflammatory |
| | | | reactions |
| 627 | Lip. 2 | 0 | normal, very slight |
| | | | inflammatory infiltration |
| | Mean = | 0.5 | |
| 632 | Lip. 3 | 1 | 1 focus: necrosis with |
| | | | considerable leukocytic |
| | | | marginal reaction |
| 633 | Lip. 3 | 0 | unremarkable |
| | Mean = | 0.5 | |
| 637 | Lip. 4 | 0.5 | no necrosis, slight ribbon- |
| | | | like inflammatory reaction |
| 638 | Lip. 4 | 0.5 | as 637 |
| | Mean = | 0.5 | |

TABLE 4
Assessment of the impairment of the animals
after s.c. administration of various preparations

| | | Behavior | Palpation | Impres- | | |
|---|---|---|---|---|---|---|
| Exp. | Admini- | of the | consec- | sion | Histolog. | |
| No. | stration | animals | utive | final | findings | Score |
| 628 | Control | 1 | 1 | 2.5 | 3 | 7.5 |
| 629 | Control | 1 | 1 | 2.5 | 3 | 7.5 |

Mean = 7.5

| | | | | | | |
|---|---|---|---|---|---|---|
| 624 | Liposomes 1 | 0 | 0 | 0 | 0 | 0 |
| 625 | Liposomes 1 | 0 | 0.5 | 0.5 | 1 | 2 |

Mean = 1

| | | | | | | |
|---|---|---|---|---|---|---|
| 626 | Liposomes 2 | 0 | 0 | 0.5 | 1 | 1.5 |
| 627 | Liposomes 2 | 0 | 0 | 0 | 0 | 0 |

Mean = 0.75

| | | | | | | |
|---|---|---|---|---|---|---|
| 632 | Liposomes 3 | 0 | 1 | 0.5 | 1 | 2 |
| 633 | Liposomes 3 | 0 | 0.5 | 0 | 0 | 0.5 |

Mean = 1.25

| | | | | | | |
|---|---|---|---|---|---|---|
| 637 | Liposomes 4 | 0 | 1 | 0 | 0.5 | 1.5 |
| 638 | Liposomes 4 | 0 | 1 | 0 | 0.5 | 1.5 |

Mean = 1.5

### EXAMPLE 3

### Tolerability of Liposomal Formulations in Rabbits

16 male Himalayan rabbits (LPT Labor der Pharmakologie und Toxikologie, Lohndorf) with a body weight between 2.15 and 2.40 kg and an age of about 3.5 months were employed as experimental animals.

The active ingredient formulations were administered subcutaneously. Administration took place in the region of the neck of two rabbits and into the flank of a further two rabbits. Each animal received four subcutaneous injections per administration day with an interval of 2 days on study days 1, 3, 5 and 7. The volume of the formulation administered was between 0.41 and 0.5 ml/administration.

Liposomal formulations 1-4 described in Example 1 were administered. No local intolerance reactions at all were found therewith. There were no signs of edema, erythema, necrotic changes or mechanical changes (scratching).

Morphological investigations starting 24 h after the last of the four subcutaneous administration for a total of 4 days also showed no morphological changes associated with the active substance.

No clinical signs of systemic toxicity were found. The eating behavior of the rabbits was unaffected. Dermatological investigations showed no changes in the parameters thromboplastin time and activated partial thromboplastin time.

### EXAMPLE 4

### Pharmacological Activity of Liposomal Formulations in Rats

The pharmacological activity of the liposomal formulations of the invention was tested on female rats (age 7 weeks, weight 100-150 g).

15 rats in total were used and were divided into 5 groups (in each case 3 rats/group). Metastatic breast carcinoma cells BN-472 (Kort et al., J. Natl. Cancer. Inst. 72 (1984), 709-713) were implanted under the breast fat pad of the rats. The following treatment regime was started on day 3 after tumor inoculation:

| | |
|---|---|
| Group A | vehicle control, 1×/day s.c. |
| Group B | aqueous solution of the active ingredient |
| | (0.3 mg/kg), 1×/day s.c. |
| Group C | liposomal formulation 4 (PC/PG pH 8.4), |
| | 0.3 mg of active ingredient/kg, 1×/day s.c. |
| Group D | liposomal formulation 4 (PC/PG pH 8.4), |
| | 1.0 mg of active ingredient/kg, 1×/day s.c. |
| Group E | liposomal formulation 4 (PC/PG pH 8.4), |
| | 3.0 mg of active ingredient/kg, 1×/day s.c. |

The occurrence of tumor foci in the lung, in the axillary lymph nodes and in the intraperitoneal lymph nodes, and the weight of the tumors were determined.

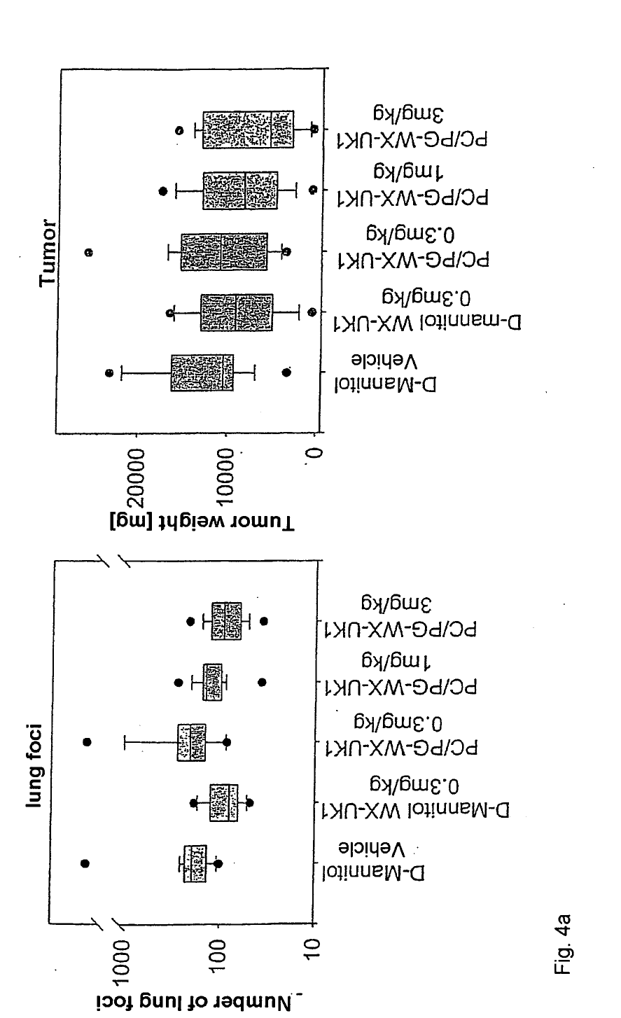

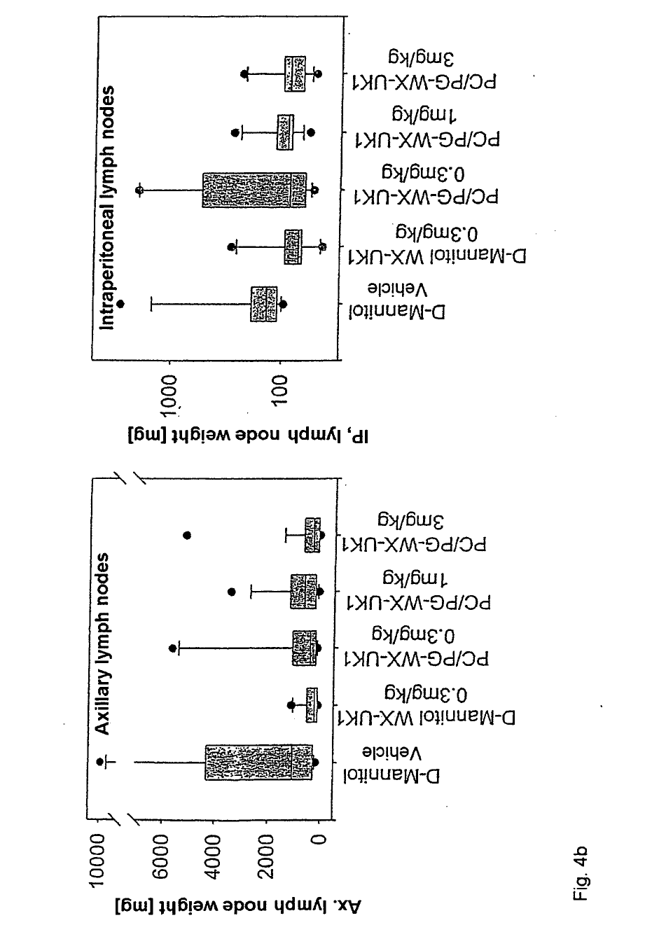

The results are depicted in FIG. 4 and show that the liposomal formulation is pharmacologically active.

### EXAMPLE 5

### Hemolysis Test with Liposomal Formulations

The intention was to test whether liposomal WX-UK1 formulations are able to reduce hemolytic properties of the active ingredient. The hemolysis test was carried out in parallel with diluted whole blood and washed erythrocytes, in each case in duplicate.

1 ml of blood (stabilized with citrate) was diluted with 4 ml of 0.9% strength NaCl solution and divided into aliquots each of 200 μl. 1 ml of the respectively tested active ingredient solution (600, 240, 120, 60, 24, 12, 0 μg/ml based on the active ingredient content) were added to the 200 μl of blood, mixed and incubated at 37° C. Incubation for 30 min was followed by centrifugation at about 1000×g for 5 min. The supernatants were removed, and the degree of hemolysis was assessed visually.

For the hemolysis test on washed erythrocytes, 1 ml of citrated blood was washed several times with 5 ml of 0.9% strength NaCl solution, and 0.4 ml of the washed erythrocytes was mixed with 4.4 ml of 0.9% strength NaCl solution. The resulting mixture was divided in aliquots each of 200 μl and otherwise treated like whole blood.

The liposomal formulations tested were formulations 1 (EPC, pH 6.5) and 2 (PC/PG, pH 6.5) according to Example 1. Only with the highest concentration (500 μg) of formulation 1 was a weak hemolysis found in whole blood. All the higher dilutions were non-hemolytic. Formulation 2 was not hemolytic for any of the tested concentrations with whole blood or washed erythrocytes.

Compared with non-liposomal formulations, an at least 10-fold reduction in the hemolytic effect was found for formulation 1 and a considerably larger reduction was found for formulation 2.

### EXAMPLE 6

### Determination of the Concentration of Free, Non-Liposomally Complexed Active Ingredient WX-UK1 in Liposomal Formulations

Formulations 1-4 according to Example 1 were tested.

60 μl of the formulation were centrifuged through a Microcon YM-50 filter unit and then investigated for the content of remaining active ingredient in an HPLC with the aid of a calibration line.

The content of free WX-UK1 in the corresponding formulations is:

| | |
|---|---|
| Formulation 1 (EPC, pH 6.5): | 550 μg/ml |
| Formulation 2 (PC/PG, pH 6.5): | 42 μg/ml |
| Formulation 3 (EPC, pH 8.4): | 461 μg/ml |
| Formulation 4 (PC/PG, pH 8.4): | 39 μg/ml |

1-27. (canceled)

28. A pharmaceutical liposomal formulation, characterized in that it comprises as active ingredient a 3-amidino- or 3-guanidino phenylalanine derivative which is effective as urokinase inhibitor, where the active ingredient is present in a proportion by weight of 0.5-100 based on the total weight of the formulation.

29. The formulation as claimed in claim 28, characterized in that the urokinase inhibitor is selected from Na-(2,4,6-triisopropylphenyl sulfonyl)-3-amidino-(D,L)-henylalanine-4-ethoxy carbonylpiperazide, the L enantiomer thereof or a pharmaceutically suitable salt of these compounds.

30. The formulation as claimed in claim 28, characterized in that the urokinase inhibitor is selected from Na-(2,4,6-triisopropylphenyl sulfonyl)-3-guanidino-(D,L)-phenylalanine-4 ethoxycarbonylpiperazide, the L enantiomer thereof or a pharmaceutically suitable salt of these compounds.

31. The formulation as claimed in claim 28, characterized in that the active ingredient is present in a proportion by weight of 2-50.

32. The formulation as claimed in claim 28, characterized in that it has a pH in the range 5.5-9.0.

33. The formulation as claimed in claim 28, characterized in that it comprises phospholipids in a proportion by weight of 4.5-400 based on the total weight of the formulation.

34. The formulation as claimed in claim 28, characterized in that it comprises phospholipids selected from neutral phospholipids, anionic phospholipids and combinations thereof.

35. The formulation as claimed in claim 28, characterized in that it comprises at least one anionic phospholipids such as, for example, phosphatidylethanolamine, phosphatidylglycerol, diphosphatidylglycerol, phosphoinositol or esterified derivatives thereof.

36. The formulation as claimed in claim 34, characterized in that it comprises phosphatidylcholine and dimyristoylphosphatidyl glycerol in a ratio of 70:30 by weight.

37. The formulation as claimed in claim 28, characterized in that it additionally comprises a membrane-stabilizing component such as, for example, cholesterol, in a proportion by weight of up to 5% based on the total weight of the formulation.

38. The formulation as claimed in claim 28 characterized in that it additionally comprises a cryoprotectant.

39. The formulation as claimed in claim 38, characterized in that the cryoprotectant is present in a proportion by weight of up to 150, preferably 5-150, based on the total weight of the formulation.

40. The formulation as claimed in claim 38, characterized in that the cryoprotectant is selected from carbohydrates or/and sugar alcohols.

41. The formulation as claimed in claim 28, characterized in that the average diameter of liposomes is not greater than 500 nm.

42. The formulation as claimed in claim 41, characterized in that the average diameter of liposomes is 100-200 nm.

43. The formulation as claimed in claim 28, characterized in that the liposomes are unilamellar liposomes.

44. The formulation as claimed in claim 28, for parenteral administration.

45. The formulation as claimed in claim 44 for intravenous injection.

46. The formulation as claimed in claim 44 for infusion.

47. The formulation as claimed in claim 44 for subcutaneous injection.

48. The formulation as claimed in claim 44 for subcutaneous injection

49. The formulation as claimed in claim 28 in dehydrated form.

50. The formulation as claimed in 28 for controlling urokinase-associated disorders.

51. The formulation as claimed in claim 50 for controlling tumors.

52. The formulation as claimed in claim 51 for controlling carcinomas of the breast, pancreatic carcinomas or/and the formation of metastases.

53. The use of a formulation as claimed in claim 28 in combination with cytostatic agents.
